(19) United States
(12) Patent Application Publication
(10) Pub. No.: US 2009/0075396 A1
(43) Pub. Date: Mar. 19, 2009

(54) Biosensors

(75) Inventors: Philip R. Deshong, College Park, MD (US); Douglas S. English, College Park, MD (US); Daniel C. Stein, College Park, MD (US); Mridula Kadalbajoo, College Park, MD (US); Fehmi Damkaci, College Park, MD (US)

Correspondence Address: VENABLE LLP, P.O. BOX 34385, WASHINGTON, DC, 20043-9998, US

(73) Assignee: University of Maryland Office of Technology Commercialization, College Park, MD (US)

(21) Appl. No.: 12/232,075

(22) Filed: Sep. 10, 2008

Related U.S. Application Data
(62) Division of application No. 11/214,012, filed on Aug. 30, 2005, now abandoned.
(60) Provisional application No. 60/605,649, filed on Aug. 30, 2004.

(51) Int. Cl. G01N 33/553; G01N 33/552; G01N 33/551

(52) U.S. Cl. 436/525; 436/527; 436/524; 977/902

(57) ABSTRACT

Methods for detecting a biological interaction comprising administering a substrate comprising a ligand attached to the substrate wherein the ligand binds to a receptor and wherein a signal is produced. Also disclosed is a biosensor comprising a substrate and a ligand wherein the ligand is attached to the surface of the substrate and wherein the ligand preferentially binds to a receptor.

### CROSS REFERENCE TO RELATED APPLICATION

The present application claims benefit under 35 U.S.C. § 119 to U.S. Provisional Patent Application No. 60/605,649 filed Aug. 30, 2004, the disclosure of which is hereby incorporated by reference in its entity.

### STATEMENT REGARDING FEDERALLY SPONSORED RESEARCH

The present invention was made with government support under Grant Nos. CA 82169-01 and, therefore, the government has certain rights in the invention.

### FIELD OF THE INVENTION

The present invention relates to methods and compositions for use as biosensors and, more particularly, the present invention includes methods and compositions for detecting a biological interaction.

### BACKGROUND OF THE INVENTION

Advances in nanoscience and engineering have led to the development of novel organic and inorganic platforms where size, size distribution, porosity, geometry and surface functionality can be controlled in the nanoscale. Nanoplatforms include dendrimers, nanoshells, quantum dots, and other inorganic particles. Several such particles are highly biocompatible and can be tailored to specific geometries such as a cylinder or a sphere. Inorganic nanoparticles generally provide a higher degree of control over size, size distribution and functionalization compared to polymeric systems such as dendrimers or nanoshells where there are inherent challenges of polymerization techniques.

The use of gold colloids in biological applications began in 1971 when Faulk & Taylor invented the immunogold staining procedure. Since that time, the labeling of targeting molecules, such as antibodies, with gold nanoparticles has revolutionized the visualization of cellular components by electron microscopy. Hayat M. Colloidal Gold: Principles, Methods and Applications, Academic, San Diego, 1989.

Gold particles display several features that make them well suited for biomedical applications including straightforward synthesis, stability and facile ability to incorporate secondary tags such as peptides targeted to specific cell types to afford selectivity. The optical and electron beam contrast properties of gold colloid have provided excellent detection capabilities for several applications, including immunoblotting, flow cytometry, and hybridization assays. Recent research involving gold nanoparticles as transfection vectors, Sandhu K K, et al., Bioconjug Chem 2002; 13: 3-6. 32 O'Brien J, et al., Brain Res Brain Res Protoc 2002; 10: 12-5; DNA binding agents, McIntosh C M, et al., J Am Chem Soc 2001; 123: 7626-9, Wang G, et al., Anal Chem 2002, 74: 4320-7; protein inhibitors, Fischer N O, et al., Proc Natl Acad Sci USA 2002, 99: 5018-23; and spectroscopic markers, Park S J, et al., Science 2002; 295: 1503-6, Weizmann Y, et al., Analyst 2001, 126: 1502-4, demonstrates the versatility of these systems in biological applications.

Gold nanoparticles have also found new applications in treating tumors using near infrared mediated radiotherapy Brongersma M. L., Nat Mater 2003; 2: 296-7. Attachment of the tumor necrosis factor (TNF) to colloidal gold nanoparticles increases tumor localization, maximizing its anticancer action while minimizing its toxicity. Combination delivery of TNF and paclitaxel using gold nanoparticles as platforms has demonstrated a higher degree of efficacy relative to free drugs Paciotti G F, et al., Drug Deliv 2004; 11: 169-83. Thus, gold nanoparticles show promise as carriers for targeted delivery to solid tumors.

Due to their inherent magnetic properties, iron oxide particles have also been a subject of intense investigation for their use as diagnostic agents. For example, detection of iron particles distributed in biological systems by magnetic resonance techniques, and other approaches to determine tumor blood flow are becoming widespread. Anzai Y, Top Magn Reson Imaging 2004, 15: 103-11. Iron oxides under study include Fe₂O₃ (maghemite), or Fe₃O₄ (magnetite). Some of the properties of iron particles include: (a) biocompatibility; (b) “imagability” by magnetic resonance imaging techniques (MRI); (c) superparamagnetic behavior (i.e., they do not retain any magnetism once the magnetic field is removed and hence under normal conditions are biologically inert to any cellular or particle-particle interactions); (d) ability to control particle size range typically to less than 100 nm so that they are efficiently removed through extravasation and renal clearance; and (e) the ability to tailor surface chemistry for colloidal stability as well as for the attachment of bioactive moieties.

Superparamagnetic nanoparticles have been widely used as MRI contrast agents enabling in vivo imaging at near microscopic resolution. Johnson G A, et al., Magn Reson Q 1993, 9: 1-30; 50 Lewin M, et al., Nat Biotechnol 2000, 18: 410-4. Magnetic nanoparticles have also found applications in cellular labeling for in vivo cell separation by MRI, as well as, for detection of early cellular apoptosis with relatively high spatial resolution. Yeh T C, et al., Magn Reson Med 1993, 30: 617-25; Zhao M, et al., Nat Med 2001, 7: 1241-4. A variety of ligands including monoclonal antibodies have been conjugated to magnetic nanoparticles to monitor cellular processes such as receptor mediated endocytosis or phagocytosis. Weissleder R, et al., J Magn Reson Imaging 1997; 7: 258-63. Dextran coated superparamagnetic nanoparticles conjugated with membrane translocating signal peptides (e.g. HIV-1 Tat protein) have been used to monitor cellular as well as nuclear trafficking and subsequent gene expression by MRI. Berry C C, et al., Int J Pharm 2004, 269: 211-25; Zhao M, et al., Bioconjug Chem 2002, 13: 840-4.

Examples of drug delivery applications of magnetic nanoparticles include PEG modified particles for uptake by mouse macrophages and breast cancer cells in vitro. Zhang Y, et al, Biomaterials 2002, 23: 1553-61; Yamazaki M, et al., Biochemistry 1990, 29: 1309-14. In addition, doxorubicin conjugated magnetic albumin nanoparticles have been used in vivo tumor therapy. Widder K J, et al., Cancer Res 1980; 40: 3512-7; Gallo J M, et al., J Pharmacokinet Biopharm 1989, 17: 305-26. The unique properties of magnetic particles described above demonstrate the potential of utilizing these agents as platforms for tumor imaging as well as targeted drug delivery.

The synthesis of ceramic nanoparticles, mostly but not exclusively based on silica, has been extensively reported, but their application in drug delivery has not been fully exploited. Ceramic particles have a number of advantages over organic polymeric particles. For example, the preparative processes involved require simple, ambient temperature conditions. The particles can be prepared with the desired size, shape, and porosity, and are extremely stable. Their small size (less than 50 nm) can allow evasion of capture by the reticuloendothelial system. In addition, there are no swelling or porosity changes with changes in pH, and they are not vulnerable to microbial attack. Silica-based particles are also known for their biocompatibility and ease of surface modification for attaching targeting ligands, drugs and imaging agents. Lal M, et al., Chem Mater 2000; 12: 2632-9. Silica based nanoparticles have been used as carriers of photosensitizing drugs for applications in photodynamic therapy. Roy I, et al., J Am Chem Soc 2003, 125: 7860-5.

Recently Martin and coworkers have demonstrated the fabrication of silica nanotubes by template synthesis and the differential functionalization of inner vs. outer surface. Mitchell D T, et al., J Am Chem Soc 2002, 124: 11864-5. The template synthetic strategy provides almost monodisperse size distribution in the fabricated nanotube dimension. Nanotubes provide the advantage over nanospheres in that their inner voids can be used for loading large amounts of drug molecules. Differential functionalization can allow the differential attachment of moieties to the inside (e.g., drugs or imaging agents) and outside (e.g., targeting moieties, antifouling agents, etc.).

Functionalization of nanoparticle surfaces with biomolecules such as DNA and proteins have been widely studied and shown to provide biosensors with many applications. A. J. Haes, et al., J. of Fluorescence, 14, 355-67, (2004); L. Jespers, et al., Protein Engineering, Design & Selection, 17, 709-13, (2004); J. Liu et al., J. of Fluorescence, 14, 343-54, (2004); V. H. Perez-Luna, et al., Encyclopedia of Nanoscience and Nanotechnology, 2, 27-49, (2004); L. A. Bauer, et al., J. of Materials Chemistry, 14, 517-26, (2004); A. J. Haes et al., Analytical and Bioanalytical Chemistry, 379, 920-30, (2004); R. Jelinek et al., Chemical Reviews, 104, 5987-6015, (2004); H. Kimura-Suda, et al., Abstracts of Papers, 226th ACS National Meeting, New York, N.Y., United States, Sep. 7-11, 2003, COLL-022, (2003). Among the nanoparticles, Au and CdSe have been most extensively investigated. X. Gao et al., Nanobiotechnology, 343-52, (2004); M. E. Flatte, Introduction to Nanoscale Science and Technology, 315-25, (2004); and A. B. Denison et al., Introduction to Nanoscale Science and Technology, 183-95, (2004).

Biomolecules, such as, oligosaccharides and glycoconjugates (glycolipids and glycoproteins) have a crucial role in inflammation, immune response, metastasis, fertilization and many other biomedically important processes. In particular, glycoproteins have important roles in cell recognition, cell adhesion and cell growth regulation.

Glycoproteins are divided into two groups that are differentiated by the type of linkage between the carbohydrate and the protein, viz. N-glycosidic glycoproteins and O-glycosidic glycoproteins.

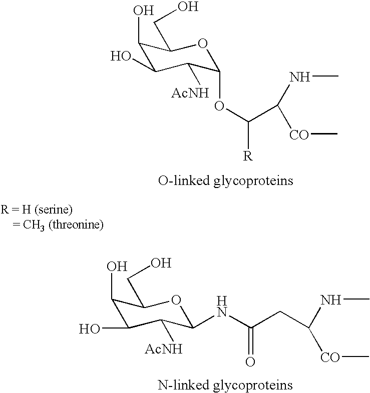

The most important step of any synthesis of a glycopeptide is the introduction of a carbohydrate residue to the amino acid in a stereoselective manner. One of the methods to make the β-N glycosidic linkage between N-acetylglucosamine and asparagine which is characteristic of N-glycoproteins is by the condensation of N-protected aspartic acid monoesters and 2-acetamido-3, 4, 6-tri-O-acetyl-2-deoxy-β-D-glucopyranosylamine in the presence of a coupling reagent like dicyclohexylcarbodiimide (DCC) (Scheme 1).

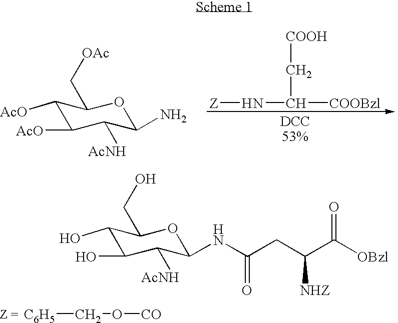

Glycosylamines can be synthesized from the reaction of an unprotected carbohydrate with aqueous ammonium hydrogen carbonate or the catalytic reduction of the corresponding azide using Pd, Lindlar catalyst, PtO₂, Raney Ni, Al/Hg, or 1, 3-propanedithiol.

Another commonly employed strategy to synthesize β-N glycosidic bonds is using glycosyl azides which can be easily prepared in high stereoselectivity and high yields (80-95%). The starting materials for the synthesis of glycosyl azides are typically halides, acetates, oxazolines or glycals. Using a glycosyl acetate, oxazoline, or glycal as a precursor provides only β-glycosyl azide, while using β- or α-glycosyl halides can provide both α- or β-glycosyl azides, respectively, e.g., Scheme 2.

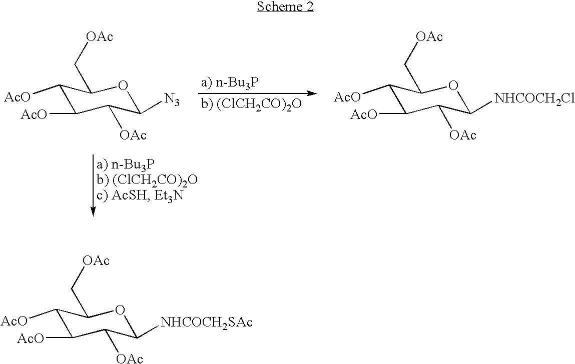

Additionally, the classical Staudinger reaction may be used which is a two step process involving the initial electrophilic addition of an azide to a trialkyl or triaryl phosphine followed by nitrogen elimination from the intermediate phosphazide to give the iminophosphorane, as shown in Scheme 3. The addition is not hindered by the substituents at phosphorus, and its rate is controlled by the inductive influence of the substituents and by the azide electrophilicity. Usually, the imination proceeds smoothly, almost quantitatively, without the formation of any side products.

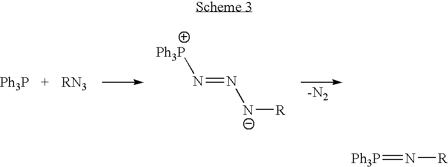

In the reaction of a glycosylazide with a trialkyl/aryl phosphine the glycosylphosphazene intermediate is known to anomerize via an open-chain structure (Scheme 4).

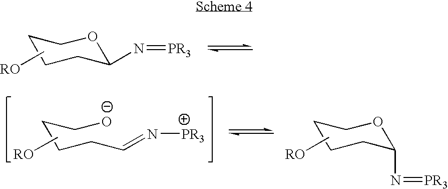

A methodology which allows for the preparation of glyconanoparticles with biologically significant oligosaccharides as well as with differing carbohydrate density has been developed by Penad\tilde{e}s et al. Penad\tilde{e}s et al., S. Chem. Eur. J. 2003, 9, 1909-1921. The approach provides water-soluble monolayer protected gold nanoclusters. The particles are prepared by in situ reduction of a gold salt in the presence of excess of the corresponding thiol-derivatized neoglycoconjugate. The mild conditions and moderate reducing agents used in this process are compatible with a wide range of ligand functionalities. The size of the nanoparticle can be controlled through the stoichiometry of the metal salt to the capping ligand (Scheme 5).

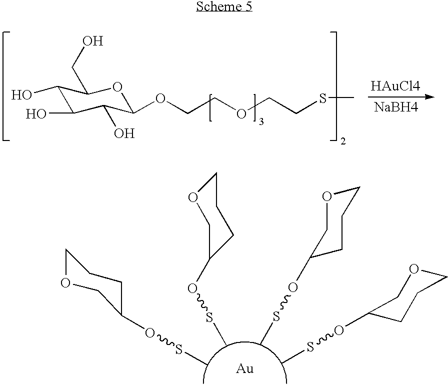

The gold nanoparticles were functionalized with the monosaccharide glucose, disaccharide lactose and maltose or trisaccharide Lewis X antigen and characterized using ¹H NMR, UV, IR and TEM, which showed clear differences related to the sugar protected clusters. These glyconanoparticles provide a glycocalyx like surface with a globular shape and well defined structure which makes them a promising tool for biological and biotechnological applications. Also, size and pattern arrangement of the metallic cluster could be controlled by using this methodology.

The disclosures of all references cited herein are hereby incorporated by reference in their entirety.

### SUMMARY OF THE INVENTION

The present invention relates to methods and compositions for use as biosensors and, more particularly, the present invention includes a methods and compositions for detecting a biological interaction.

In an exemplary embodiment, the present invention includes a method of detecting a biological interaction comprising administering a substrate comprising a ligand wherein the ligand is attached to the substrate and binds to a receptor and wherein a signal is produced.

In a preferred embodiment, the present invention includes a method of detecting a biological interaction comprising administering a gold nanoparticle comprising a ligand wherein the ligand is attached to the gold nanoparticle and binds to a receptor and wherein at least one gold nanoparticle becomes luminescent.

In another exemplary embodiment, the present invention includes a biosensor comprising a substrate and a ligand wherein the ligand is attached to the substrate and wherein the ligand binds to a receptor to produce a signal.

In a preferred embodiment, the present invention includes a biosensor comprising a gold nanoparticle and a ligand wherein the ligand is attached to the gold nanoparticle and wherein the ligand binds to a receptor wherein the gold nanoparticle becomes luminescent.

### BRIEF DESCRIPTION OF THE DRAWINGS

FIG. 1A shows an AFM image of a SAM from β-glucose thiol conjugate;

FIG. 1B shows an AFM image of a SAM from α-glucose thiol conjugate;

FIG. 2 shows the synthetic scheme for gold nanoparticle surface functionalization, in which X and Y are same or different biomolecules and represent targeting moiety of the substrate and wherein attachment of a molar mixture of symmetric or asymmetric disulfides of varying content can be achieved on the surface;

FIG. 3 shows possible multidentate binding process of oligosaccharide-based biosensors;

FIG. 4 shows a comparison of the luminescence detected from: (A) Neisseria gonorrhoeae; (B) Neisseria gonorrhoeae with uncoated gold nanoparticles; (C) Neisseria gonorrhoeae with glucosylated nanoparticles; and (D) Neisseria gonorrhoeae with lactosylated nanoparticles;

FIG. 5 shows (A) a TEM image of concanavalin A mediated aggregation of glucose-coated Au nanoparticles and (B) a photoluminescence image of aggregated particles;

FIG. 6 shows AFM images of concanavalin A binding to thiol-glucose patterned strips.

### DETAILED DESCRIPTION

The present invention relates to methods and compositions for use as biosensors. According to some embodiments, the present invention includes a method of detecting a biological interaction comprising administering a substrate comprising a ligand wherein the ligand is attached to the substrate and binds to a receptor and wherein a signal is produced. According to other embodiments, the present invention includes a biosensor comprising a substrate and a ligand wherein the ligand is attached to the surface of the substrate and wherein the ligand preferentially binds to a receptor.

The methods and compositions of the present invention may be used as biosensors in a variety of applications. For example, the methods and compositions may be used to detect for any known pathogenic e-coli, bacteria or virus. In other examples, the methods and compositions may be used for the detection of biotoxins. In yet other examples, the methods and compositions may be used for the detection of enzymes. In still other examples, the methods and compositions may be used for the detection of lectins (e.g., ricin).

The possible ligands that may be attached to a substrate and receptors according to the present invention will be readily apparent by one skilled in the art upon reading the present disclosure. For example, possible receptors and substrates according to the present invention are disclosed in, e.g., Kurosh et al., Gene and Cell Therapy, 2nd Edition, 223-244 (2004); Uner et al., Neoplasma, 51:4, 269-274 (2004); Taitt, C. et al., Microbial Ecology, 47:2, 175-185 (2004); Rajcani, J., Microbial, Algal, and Fungal Biochemistry, 50:4, 407-431 (2003); Tailor, S. et al., Microbial, Algal, and Fungal Biochemistry, 281, 29-106 (2003); and Gallo, S. et al., Biochimica et Biophysica Acta, 1614:1, 36-50 (2003), the disclosures of which are hereby incorporated by reference.

In some embodiments, the methods and compositions of the present invention may be used as in vivo biosensors. For example, the present invention includes methods of detecting a biological interaction in a subject in which a substrate comprising a ligand is administered to the subject and wherein the ligand binds to a receptor to produce a signal. In other examples, the methods and compositions of the present invention may be used as in vitro biosensors. In some examples, a sample of bodily fluid is taken from a subject and a substrate comprising a ligand is administered to the sample wherein if the ligand binds to a receptor a signal is produced. In other examples, the methods and compositions of the present invention may be used to detect for the presence of substances (e.g., ricin) in, for example, drinking water.

Gold nanoparticles coated with a lactose analog have been shown to bind selectively to the lactose receptor on endothelial cells including Neisseria sps and other bacteria.

The methods and compositions according to the present invention include a substrate. In some embodiments, the substrate may comprise gold, silver, silica, iron oxide, platinum, CdSe and combinations thereof. In further embodiments, the substrate may be a particle or a film. In preferred embodiments the substrate is a nanoparticle.

In preferred embodiments, substrates according to the present invention include gold nanoparticles. Gold nanoparticles have characteristics that make them ideal for the development of diagnostics in biological systems. For example, gold nanoparticles are non-toxic and can be employed for in vivo studies. In addition, individual gold nanoparticles and colloidal gold nanoparticles of >1 nm in diameter are not luminescent. In contrast, colloidal gold nanoparticles with diameters of <1 nm are highly luminescent. Gonzalez, et al., Physical Review Letters, 93, 147402/11-4, (2004); J. Zheng, et al., Physical Review Letters, 93, 077402/1-/4, (2004), the disclosures of which are hereby incorporated by reference in their entirety. Thus, the methods and compositions of the present invention may be used to produce luminescent particles (e.g., gold) comprised of an aggregate of small particles. Furthermore, gold and CdSe nanoparticles are highly fluorescent, but unlike typical fluorescent molecules, are not prone to photobleaching. In addition, CdSe particles can be prepared in to provide many wavelengths of light based on the size of the particle.

The methods and compositions according to the present invention also include a ligand attached to the substrate. The ligand may include any molecule that specifically binds to a receptor of interest. In some examples, more than one ligand may be attached to the surface of the substrate to allow binding to more than one receptor or to bind to a single receptor. In one exemplary embodiment the ligand may include one or more antibodies to detect an antigen of interest. In another exemplary embodiment, the ligand may include a nucleotide sequence (e.g., DNA or RNA). In another example, the ligand may include a protein. In yet another example, the ligand may include a saccharide. In still another example, the ligand may include a glycoprotein.

In a preferred embodiment, the ligand is an oligosaccharide. Oligosaccharides play critical roles in a variety of biological processes in eukaryotic cells including cell-cell recognition, cell-cell signaling, modulating cell growth and intracellular trafficking of proteins. For example, glycosyl-based cell surface receptors have been implicated in cell fertilization, invasion of host cells by pathogens, and tumor metastasis. Glycosyl-based ligands may be used in the methods and compositions of the present invention due to its high specificity of the binding process.

In another preferred embodiment the ligand is a glycoprotein. Due to their importance in cellular mediation processes, there has been tremendous interest in the synthesis of N-linked glycoprotein linkages. Novel methods for the synthesis of N-linked glycoproteins for use in the present invention may include using glycosyl azides (Scheme 6).

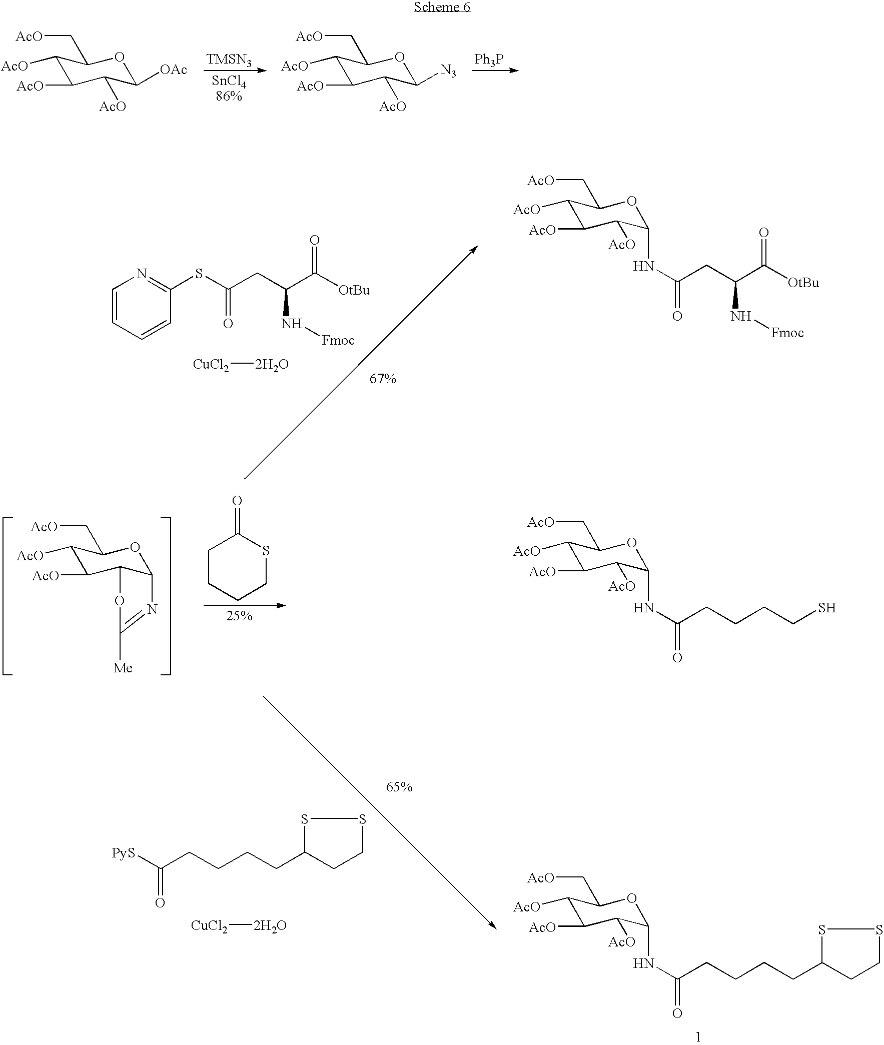

The methods according to the present invention have two significant advantages over previous methods: (1) the synthesis of oligosaccharide azide precursors can be prepared from intact, biologically relevant, complex oligosaccharide derivatives, and (2) the key coupling reaction can be performed on complex peptide derivatives. This methodology has been employed to conjugate a variety of mono-, di-, and trisaccharide derivatives to aspartic acid and short peptide derivatives. Thus, the method of scheme 6 may be used to prepare ligands for a variety of biosensors. For example, this method for the synthesis of oligosaccharide conjugates may be used to prepare ligands as oligosaccharide based cell surface receptors in many biological processes. Consequently, due to the generality of the methods described herein and known in the art, the preparation of receptors that may be used for known pathogens may be readily prepared.

In some embodiments of the present invention, the synthesis methods described above may be used to produce oligosaccharide conjugates that are attached to a substrate, for example, gold nanoparticles or gold surfaces. Moreover, according to the methods and compositions of the present invention a single oligosaccharide receptor or multiple oligosaccharide receptors may be attached to the substrate.

For example, oligosaccharide coupling on gold surfaces or gold nanoparticles may be used to form self-assembled monolayers (SAMs). Using Atomic Force Microscopy (AFM) it has been shown that the density of surface coverage on a gold surface depends on the nature of the oligosaccharide.

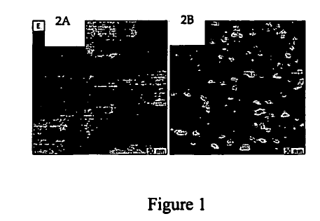

In an exemplary embodiment, a gold substrate may be used for the attachment to a ligand comprising a thiol or a disulfide. SAMs derived from the oligosaccharide bioconjugates on gold (111) surfaces have been characterized using XPS, FT-IR, and AFM analysis. The oligosaccharides form ordered, dense monolayers. As shown in FIG. 1 a SAM from α-glucose thiol conjugate has “holes” in the SAM compared to its stereochemical β-glucose counterpart. Thus, it is feasible to synthesize functionalized nanoparticles with control over the coating density. Moreover by changing the sugar(s) on the gold nanoparticles, specific recognition of these functionalized nanoparticles by both enzymes and cells may be achieved. In addition, these strategies could also be applied to the preparation of substrates including nucleic acids (e.g., DNA, RNA) or proteins.

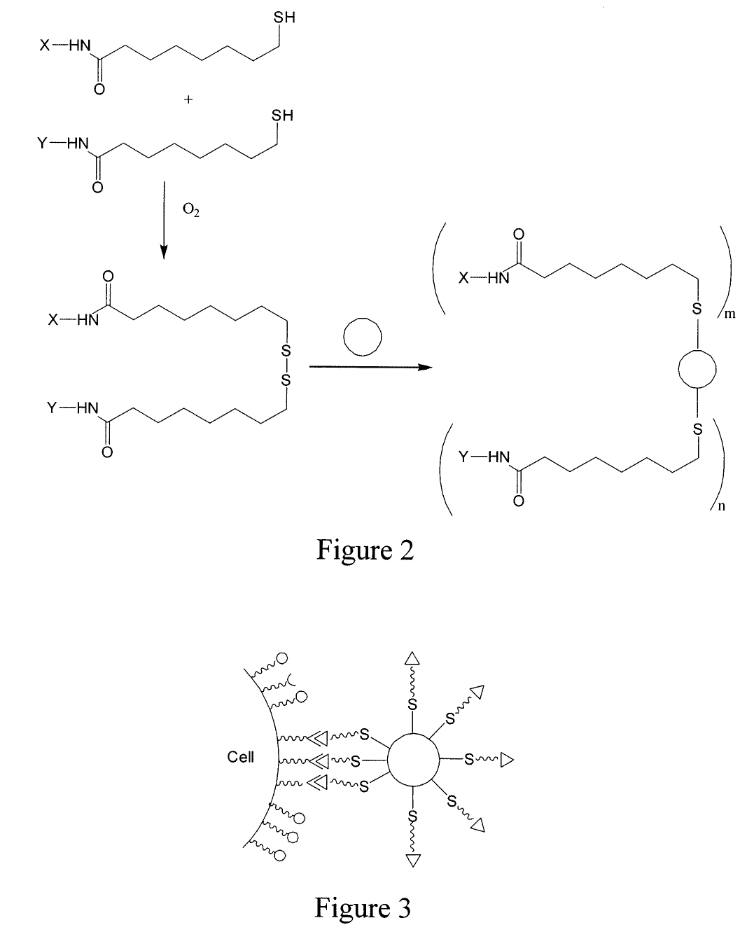

In preferred embodiments the ligand is attached to the surface of the substrate. For example, surface functionalization may employ first the conjugation of appropriate amine terminated biomolecule derivatives (drugs, targeting moieties etc.). In particular, symmetrical and unsymmetrical disulfides with general structure X—S—S—Y may be synthesized (where X and Y are either same or different biomolecules). Exposure of gold nanoparticles to appropriate molar mixture of symmetrical and unsymmetrical disulfide derivatives with different ligands attached will result in a covalent attachment of the bioconjugates to the gold surface, as shown in FIG. 2. By varying the molar ratio of the targeting moiety of the substrate, the content of these species on the gold surface can be controlled. The functionalized substrates may then be characterized by, any method known in the art, including surface-enhanced FT-IR, AFM and X-ray photoelectron spectroscopy (XPS).

The substrate may be coated with a single receptor or with multiple receptors using the technology developed. For example, an asymmetrical disulfide (ligandA-S—S-ligandB) may be used such that binding of the disulfide yields a substrate (e.g., a gold nanoparticle) with both ligands attached.

When non-luminescent nanoparticles undergo aggregation, the resulting aggregates are highly luminescent. For example, the luminescence of the coated nanoparticles increases dramatically upon binding of cells or enzymes to the coated surface. The methods and compositions of the present invention provide an efficient preparation of a variety of functionally coated nanoparticles in which both the ligand and the wavelengths of the luminescent probe could be altered.

The functionalized substrates according to the present invention may be used for the recognition of any receptor to detect, e.g., specific enzymes and cells. For example, according to the present invention oligosaccharides may be used as ligands for the recognition of biological systems (e.g. tumor cells, pathogens).

The cell recognition phenomenon for saccharide-based biologicals is different than typical protein-protein interactions, since glycosyl recognition is generally a multidentate process. Since each binding event of a glycosyl-mediated process involves weak interactions (H-bonding), the many ligand-receptor interactions are involved to achieve high specificity in surface recognition events. Accordingly, the recognition of glycosyl residues on the cell surface requires the clustering of surface receptors (see FIG. 3). It is this multidentate binding process that provides a unique advantage of oligosaccharide-based biosensors have over other biomolecules, i.e., proteins or nucleic acids. A biosensor based on oligosaccharide binding should be able to present a multidentate display of glycosyl residues to the cell surface. Nanoparticles coated with oligosaccharides are ideally suited for this sort of biosensor since they have a large number of ligands displayed in all directions and can readily provide multidentate binding.

In further embodiments of the present invention, the frequency of luminescence of the biosensor may be modified to produce a desired luminescence. For example, by changing the distance between the particles, the emission spectrum of the aggregate may be modified. In other examples, the size of the substrate may be changed to modify the emission spectrum. Accordingly, by changing the size and size-distribution of particles used as substrates and/or the ligand, the emission spectrum of a biosensor could be controllably modified to change the frequency and/or wavelength of luminescence. For example, the biosensor could be controllably modified to shift the wavelength of luminescence to about 800 nm. Accordingly, the present invention may be used to provide tunable optical signals.

The methods and compositions of the present invention can be used as biosensors in a variety of applications. For example, the methods and compositions may be used to detect for any known pathogenic e-coli, bacteria or virus. In other examples, the methods and compositions may be used for the detection of enzymes. In still other examples, the methods and compositions may be used for the detection of lectins (e.g., ricen). In addition, use as biosensors. Gold nanoparticles coated with a lactose analog have been shown to bind selectively to the lactose receptor on endothelial cells including Neisseria sps and other bacteria.

To facilitate a better understanding of the present invention, the following examples of some of the preferred embodiments are given. In no way should such examples be read to limit, or define, the scope of the invention.

### EXAMPLES

Nanografting and microcontact printing techniques have been used to fabricate gold surfaces with features of 10 nm to 10 micrometers that are coated with the oligosaccharide conjugates. The resulting gold surfaces have been shown to bind specifically to lectins and cells with complimentary receptor sites. Gold nanoelectrodes have also been coated with oligosaccharide conjugates. The resulting electrodes detect the specific binding to the oligosaccharides by both enzymes and cells.

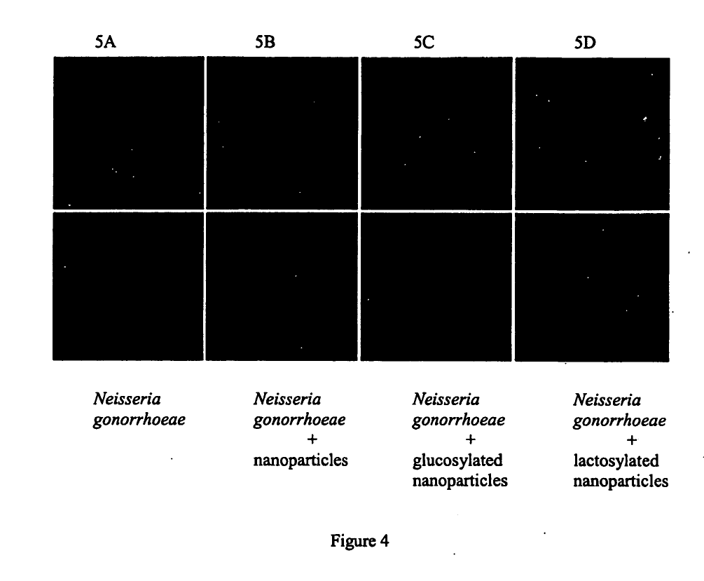

Gold nanoparticles coated with a lactose analog have been shown to bind selectively to the lactose receptor on endothelial cells including Neisseria sps and other bacteria. In particular, a strain of the pathogen Neisseria, the organism that causes gonorrhea, expresses a lactose receptor on its surface and has been shown to selectively bind to lactose-coated nanoparticles. As shown in FIG. 4D, binding of lactose functionalized gold nanoparticles to the cell surface receptors of Neisseria gonorrhoeae leads to dramatically enhanced luminescence from the gold particles aggregated on the surface of the cell. In contrast, Neisseria gonorrhoeae cells alone (FIG. 4A), Neisseria gonorrhoeae cells in the presence of gold nanoparticles (FIG. 4B), and glucose-coated nanoparticles in the presence of Neisseria gonorrhoeae (FIG. 4C) did not display enhanced luminescence, which is indicative of binding. The control experiments demonstrate that the binding is highly specific only for the lactose conjugate. The selective binding of these functionalized nanoparticles with a concomitant increase in luminescence may, therefore, be used as an assay for Neisseria detection. Thus, the present invention provides novel methods and compositions that may be used as cell specific biosensors.

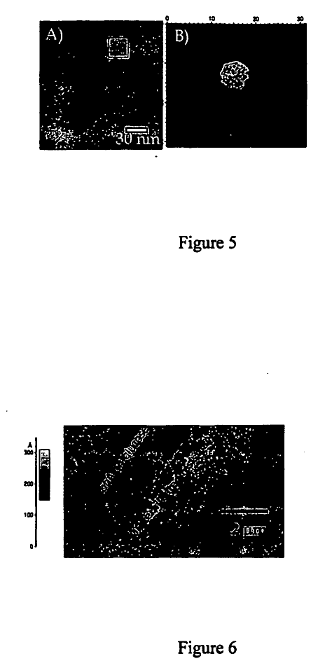

In addition, it has been shown that non-luminescent Au nanoparticles functionalized with cell surface receptors become luminescent upon lectin-induced aggregation. FIG. 5 shows both TEM and luminescence images of aggregated particles mediated by the binding of concanavalin A (con A), a lectin protein specific to glucose.

FIG. 6 illustrates the specificity of con A to thiol-glucose derivatives. In this example thiol-glucose derivatives were micro patterned on a gold substrate and allowed to react with con A in solution which yielded the raised regions shown in FIG. 6. Two con A proteins serve as “spacers” between nanoparticles. X-ray analysis of con A has shown that the unit cell of the monomer is ˜2 nm in every dimension. Since the Au nanoparticle aggregates are held together as dimers, the TEM images confirmed that the particles aggregate with a “spacer” dimension of ˜6 nm between each particle (2 molecules of con A between each particle contribute 4 nm and the oligosaccharide chain contribute 1 nm twice).

By varying either the length of the ligand or the lectin employed to induce aggregation, it may be possible to vary the distance between particles in a systematic manner, and alter the emission of the aggregates. For example, aggregation with a lectin significantly larger than con A may cause the nanoparticles in the aggregate to be separated by a distance >4 nm. Thus, by changing the distance between the particles, the emission spectrum of the aggregate would also be changed. Furthermore, by changing the size of the substrate particles the emission spectrum may similarly be altered. Accordingly, by changing the size and size-distribution of particles used as substrates and/or the ligand used, the emission intensity or wavelengths of the biosensor may be controllably modified to change the frequency of luminescence. Accordingly, the present invention may be used to provide tunable optical signals.

Gold nanoparticles of 5 nm diameter have also been prepared and linked to oligosaccharides having thiol and disulfide side chains characterized using transmission electron microscopy (TEM). TEM experiments of bare (non-ligated) gold nanoparticles in a solution of CH₂Cl₂ showed that the particles were randomly dispersed. Gold nanoparticles were then attached with α-glu-OH—SH. The TEM images showed some amount of clustering of the gold nanoparticles. There is a certain extent of hydrogen-bonding between the hydroxylated sugar units, which caused aggregation of the gold nanoparticles. Next concanavalin-A, a plant lectin, which binds specifically to manno- and glucopyranosides, bound to gold nanoparticles were prepared and it was shown that the clustering of the gold nanoparticles was greatly enhanced.

In addition, the effect of changing the gold to sugar molar ratio was investigated to determine if clustering of the nanoparticles would be effected. Lowering the Au:sugar molar ratio showed that although some aggregation of the gold nanoparticles was observed, there were also some free particles. This experiment showed that con A enhanced the clustering of the gold nanoparticles. In addition, it was shown that dilution of the sugar solutions with ethanol had no effect on the clustering of the particles.

It is known that con A exists as a dimer below pH 7 and as a tetramer above pH 7. To investigate the effect of pH on nanoparticle aggregation, TEM experiments of a solution of α-glu-OH—S₂, Au nanoparticles and con A at a pH of 5 were performed. The aggregation of the nanoparticles reduced significantly at pH 5, as expected, since con A exists as a dimer rather than a tetramer at this pH.

The above examples demonstrates that the present invention is well adapted to carry out the objects and attain the ends and advantages mentioned as well as those that are inherent therein.

While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure. The depicted and described embodiments of the invention are exemplary only, and are not exhaustive of the scope of the invention. Consequently, the invention is intended to be limited only by the spirit and scope of the appended claims, giving full cognizance to equivalence in all respects. All references cited herein are hereby incorporated by reference in their entirety.

1. A method of detecting a biological interaction comprising:
providing a substrate comprising a ligand which is attached to the substrate and binding the ligand to a receptor, wherein a signal is produced from the substrate; and
detecting the signal produced fiom the substrate.

2. The method of claim 1, wherein the substrate comprises gold, silver, silica, iron oxide, platinum, CdSe and combinations thereof.

3. The method of claim 2, wherein the substrate is a nanoparticle.

4. The method of claim 2, wherein the substrate is a film.

5. The method of claim 1, wherein the ligand is attached to a surface of the substrate.

6. The method of claim 1, wherein the signal comprises a luminescence from the substrate.

7. The method of claim 6, wherein the wavelength of luminescence is tunable.

8. The method of claim 1, wherein the ligand is selected fiom the group consisting of an antibody, an antigen, a nucleotide sequence, a protein, a saccharide, an oligosaccharide, a glycoprotein, a thiol, a disulfide, a vaccine or combinations thereof.

9. The method of claim 1, wherein the receptor is selected from the group consisting of an antibody, an antigen, a nucleotide sequence, a protein, a saccharide, an oligosaccharide, a glycoprotein, a thiol, a disulfide, a vaccine or combinations thereof.

10. A method of detecting a biological interaction comprising:
providing a gold nanoparticle comprising a ligand which is attached to the gold nanoparticle and binding the ligand to a receptor, wherein the gold nanoparticle luminesces; and
detecting the luminescence of the gold nanoparticle.

11. The method of claim 10, wherein the ligand is selected from the group consisting of an antibody, an antigen, a nucleotide sequence, a protein, a saccharide, an oligosaccharide, a glycoprotein, a thiol, a disulfide, a vaccine or combinations thereof.

12. The method of claim 10, wherein the receptor is selected from the group consisting of an antibody, an antigen, a nucleotide sequence, a protein, a saccharide, an oligosaccharide, a glycoprotein, a thiol, a disulfide, a vaccine or combinations thereof.

13.-26. (canceled)
